(19) United States
(12) Patent Application Publication
(10) Pub. No.: US 2022/0076784 A1
(43) Pub. Date: Mar. 10, 2022

(54) SYSTEMS AND METHODS FOR IDENTIFYING FEATURE LINKAGES IN MULTI-GENOMIC FEATURE DATA FROM SINGLE-CELL PARTITIONS

(71) Applicant: 10x Genomics, Inc., Pleasanton, CA (US)

(72) Inventors: Li Wang, Pleasanton, CA (US); Yiming Kang, St. Louis, MO (US)

(21) Appl. No.: 17/465,725

(22) Filed: Sep. 2, 2021

Related U.S. Application Data
(60) Provisional application No. 63/075,009, filed on Sep. 4, 2020.

(51) Int. Cl. G16B 20/40

(52) U.S. Cl. CPC G16B 20/40

(57) ABSTRACT

Methods and systems for generating linkage correlations and linkage significances between a first genomic feature and a second genomic feature identified for each of a plurality of cells may be provided. For example, the method may comprise receiving a data matrix comprising a first genomic feature and a second genomic feature identified for each of a plurality of cells; smoothing the data matrix to generate a smoothed matrix; generating linkage correlations between the first genomic feature and second genomic feature identified for each of the plurality of cells in the data matrix; generating linkage significances using multiplication of a plurality of linkage matrixes; and outputting the linkage correlations and linkage significances for each of the plurality of cells in the data matrix.

### CROSS-REFERENCE TO RELATED APPLICATION

The present application claims priority to and the benefit of the U.S. Provisional Patent Application No. 63/075,009, filed Sep. 4, 2020, titled “SYSTEMS AND METHODS FOR IDENTIFYING FEATURE LINKAGES IN MULTI-GENOMIC FEATURE DATA FROM SINGLE-CELL PARTITIONS,” which is hereby incorporated by reference in its entirety as if fully set forth below and for all applicable purposes.

### FIELD

The embodiments provided herein are generally related to systems and methods for analysis of genomic nucleic acids and genomic features. Included among embodiments provided herein are systems and methods relating to accurate detection of feature linkage based on analysis of more than one genomic features.

### BACKGROUND

Accurate detection of cell-associated barcodes such as, for example, barcodes from partitions containing single cells, is a primary step in the analysis of single-cell molecular datasets from barcoded partitions. Correct cell-calling remains an important challenge for the successful analysis of unbiased genome-wide single-cell molecular datasets. However, this problem becomes even more challenging when addressing the recovery of multiple genomic features. Each genomic feature introduces its own unique biochemistry, signal-to-noise profile and measurement inefficiencies. In addition, biology itself can generate diverse combinations of the levels of each genomic feature in a cell.

Deciphering the transcriptional network is one of the central tasks in biomedical research. From a signal transduction point of view, the transcriptional network initializes from the input signal delivered to the nucleus and mediated by the interactions between cis non-coding elements, such as enhancers and promoters, and transcription factors and cofactors, and is concluded as the transcription of target genes. Transcription factors bind to specific enhancers and promoters and activate the expression of target genes encoded in cis. The accessibility of enhancers for different transcription factors and the target gene pool is very context-specific, which is essential for cell-type diversity, the adaptability in tissue responding to stress, injury and pathogenesis.

As such, there is a need for better detection of a relationship between genomic features such as genes and accessibility of non-coding elements, for example, enhancers and promoters.

### SUMMARY

In accordance with various embodiments, a method is provided for generating linkage correlations and linkage significances between a first genomic feature and a second genomic feature identified for each of a plurality of cells, the method comprising receiving a data matrix comprising a first genomic feature and a second genomic feature identified for each of a plurality of cells; smoothing the data matrix to generate a smoothed matrix, wherein smoothing the data matrix comprises normalizing the first genomic feature and the second genomic feature identified for each cell in the data matrix with the first genomic feature and second genomic feature identified for each of a selected subset of neighboring cells; generating linkage correlations between the first genomic feature and second genomic feature identified for each of the plurality of cells in the data matrix; generating linkage significances using multiplication of a plurality of linkage matrixes, each linkage matrix comprising linkage correlations between the first genomic feature and the second genomic features identified for each of the plurality of cells in the data matrix; and outputting the linkage correlations and linkage significances for each of the plurality of cells in the data matrix.

In accordance with various embodiments, there may be provided a non-transitory computer-readable medium storing computer instructions that, when executed by a computer, cause the computer to perform a method for generating linkage correlations and linkage significances between a first genomic feature and a second genomic feature identified for each of a plurality of cells, the method comprising receiving a data matrix comprising a first genomic feature and a second genomic feature identified for each of a plurality of cells; smoothing the data matrix to generate a smoothed matrix, wherein smoothing the data matrix comprises normalizing the first genomic feature and the second genomic feature identified for each cell in the data matrix with the first genomic feature and second genomic feature identified for each of a selected subset of neighboring cells; generating linkage correlations between the first genomic feature and second genomic feature identified for each of the plurality of cells in the data matrix; generating linkage significances using multiplication of a plurality of linkage matrixes, each linkage matrix comprising linkage correlations between the first genomic feature and the second genomic features identified for each of the plurality of cells in the data matrix; and outputting the linkage correlations and linkage significances for each of the plurality of cells in the data matrix.

In accordance with various embodiments, a system is provided for generating linkage correlations and linkage significances between a first genomic feature and a second genomic feature identified for each of a plurality of cells, the system comprising a data store configured to store a data set at least associated with a plurality of cells, wherein the data set comprises molecule counts of at least two genomic features for each cell of a plurality of cells; and a computing device communicatively connected to the data store and configured to receive the data set, the computing device comprising a feature linkage analysis engine configured to receive a data matrix comprising the first genomic feature and the second genomic feature identified for each of a plurality of cells, smooth the data matrix to generate a smoothed matrix, wherein smoothing the data matrix comprises normalizing the first genomic feature and the second genomic feature identified for each cell in the data matrix with the first genomic feature and second genomic feature identified for each of a selected subset of neighboring cells, generate linkage correlations between the first genomic feature and second genomic feature identified for each of the plurality of cells in the data matrix, and generate linkage significances using multiplication of a plurality of linkage matrixes, each linkage matrix comprising linkage correlations between the first genomic feature and the second genomic features identified for each of the plurality of cells in the data matrix; and a display communicatively connected to the computing device and configured to display a report comprising the linkage correlations and linkage significances.

These and other aspects and implementations are discussed in detail herein. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIGS. 1A and 1B are schematic illustrations of non-limiting examples of the sequencing workflow for using single cell targeted gene expression sequencing analysis to generate sequencing data for analyzing the expression profile of targeted genes of interest, in accordance with various embodiments.

FIG. 2 is an exemplary flowchart showing a process flow for conducting sequencing data analysis, in accordance with various embodiments.

FIG. 3 is an exemplary flowchart showing a process flow for feature linkage analysis, in accordance with various embodiments.

FIG. 4 is another exemplary flowchart showing a process flow for feature linkage analysis, in accordance with various embodiments.

FIG. 5 is a schematic diagram of non-limiting examples of a system for feature linkage analysis, in accordance with various embodiments.

FIGS. 6A-6D are graphs depicting that matrix smoothing improves interpretability of the linkage correlations, in accordance with various embodiments.

FIG. 7 are plots depicting distributions for linkage correlation and significance for a 5 k peripheral blood mononuclear cell (PBMC) dataset, in accordance with various embodiments.

FIG. 8 is a block diagram of non-limiting examples illustrating a computer system for use in performing methods provided herein, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

The above-identified figures are provided by way of representation and not limitation. The figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

Provided herein are methods and systems for feature linkage analysis, particularly for detecting pairs of genomic features detected in each of a plurality of cells, such as open chromatin regions (e.g., promoters, enhancers, etc.) and genes with a significant correlation in signals across cells. Such methods and systems can be used, for example for integration of single-cell transcriptomics and epigenomics. It should be appreciated, however, that although the systems and methods disclosed herein can refer to their application in the integration of single-cell transcriptomics and epigenomics workflows, they are equally applicable to other analogous fields.

The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

It should be understood that any use of subheadings herein is for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

All publications mentioned herein are incorporated herein by reference for the purpose of describing and disclosing devices, compositions, formulations and methodologies which are described in the publication and which might be used in connection with the present disclosure.

As used herein, the phrase “genomic feature” refers to one or more defined or specified genome elements or regions. In some instances, the genome elements or regions can have some annotated structure and/or function (e.g., open chromatin regions such as a promoter, an enhancer, a fragment end or a cutsite, a chromosome, a gene, protein coding sequence, mRNA, lncRNA (long non-coding RNA), tRNA, rRNA, repeat sequence, inverted repeat, miRNA, siRNA, etc.) or can be a genetic/genomic variant (e.g., single nucleotide polymorphism/variant, insertion/deletion sequence, copy number variation, inversion, etc.) which denotes one or more nucleotides, genome regions, genes or a grouping of genome regions or genes (in DNA or RNA) that have undergone changes as referenced against a particular species or sub-populations within a particular species due to, for example, mutations, recombination/crossover or genetic drift. In some other instances, the genomic feature can be measured by cell surface protein expression, mutational status, or counts of intronic reads.

As used herein, the phrase “Assay for Transposase-Accessible Chromatin sequencing” or “ATAC sequencing” refers to a sequencing method that probes DNA accessibility with an artificial transposon, which inserts specific sequences into accessible regions of chromatin. Because the transposase can only insert sequences into accessible regions of chromatin not bound by transcription factors and/or nucleosomes, sequencing reads can be used to infer regions of increased chromatin accessibility.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within one, two, three, four, five, six, seven, nine, or ten percent.

The term “ones” means more than one.

As used herein, the term “plurality” can be 2, 3, 4, 5, 6, 7, 8, 9, 10, or more.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, un-recited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. Standard molecular biological techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and standard techniques described herein are those well-known and commonly used in the art.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

DNA (deoxyribonucleic acid) is a chain of nucleotides containing 4 types of nucleotides; A (adenine), T (thymine), C (cytosine), and G (guanine), and RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

As used herein, the term “cell” is used interchangeably with the term “biological cell.” Non-limiting examples of biological cells include eukaryotic cells, plant cells, animal cells, such as mammalian cells, reptilian cells, avian cells, fish cells or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells and the like. A mammalian cell can be, for example, from a human, mouse, rat, horse, goat, sheep, cow, primate or the like.

A term “genome’, as used herein, refers to the genetic material of a cell or organism, including animals, such as mammals, e.g., humans and comprises nucleic acids, such as DNA. In humans, total DNA includes, for example, genes, noncoding DNA and mitochondrial DNA. The human genome typically contains 23 pairs of linear chromosomes: 22 pairs of autosomal chromosomes (autosomes) plus the sex-determining X and Y chromosomes. The 23 pairs of chromosomes include one copy from each parent. The DNA that makes up the chromosomes is referred to as chromosomal DNA and is present in the nucleus of human cells (nuclear DNA). Mitochondrial DNA is located in mitochondria as a circular chromosome, is inherited from only the female parent, and is often referred to as the mitochondrial genome as compared to the nuclear genome of DNA located in the nucleus.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject.

In some examples, such systems provide “sequencing reads” (also referred to as “fragment sequence reads” or “reads” herein). A read may include a string of nucleic acid bases corresponding to a sequence of a nucleic acid molecule that has been sequenced. In some situations, systems and methods provided herein may be used with proteomic information.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM), Ion Torrent, and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The term “read” or “sequencing read” with reference to nucleic acid sequencing refers to the sequence of nucleotides determined for a nucleic acid fragment that has been subjected to sequencing, such as, for example, next generation sequencing (“NGS”). Reads can be any a sequence of any number of nucleotides which defines the read length.

Methods of processing and sequencing nucleic acids in accordance with the methods and systems described in the present application are also described in further detail in U.S. Ser. Nos. 14/316,383; 14/316,398; 14/316,416; 14/316,431; 14/316,447; and 14/316,463 which are herein incorporated by reference in their entirety for all purposes and in particular for all written description, figures and working examples directed to processing nucleic acids and sequencing and other characterizations of genomic material.

The term “barcode,” as used herein, generally refers to a label, or identifier, that conveys or is capable of conveying information about an analyte. A barcode can be attached to a support, for example, a bead, such as a solid bead or a gel bead. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include barcode sequences, such as: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing reads.

As used herein, the term “cell barcode” refers to any barcodes that have been determined to be associated with a cell, as determined by a “cell calling” step within various embodiments of the disclosure. For example, the cell barcode can be a known nucleotide sequence which serves as a unique identifier for a single cell partition, such as a single GEM droplet or well. Each cell barcode can contain reads from a single cell.

As used herein, the term “Gel bead-in-EMulsion” or “GEM” refers to a droplet containing some sample volume and a barcoded gel bead, forming an isolated reaction volume. When referring to the subset of the sample contained in the droplet, the term “partition” may also be used. In various embodiments within the disclosure when used with GEM, the term “barcode” refers to a known nucleotide sequence or a known combination of several nucleotide sequences, which serve as a unique identifier for a single GEM droplet. Each barcode usually contains reads from a single cell. For example, a barcode can comprise one, two, three, four, five, or more known barcode sequences. These barcode sequences attached to the same GEM can be the same or different, but a combination of these barcodes will be unique for the same GEM and be different from another combination of barcode sequences on different GEMs. For example, each GEM has a ATAC DNA barcode oligonucleotide and a gene expression barcode oligonucleotide attached. Although the ATAC DNA barcode oligonucleotide and the gene expression barcode oligonucleotide may be different, they are designed to have a known association, so each genomic feature receives a cell-associated barcode that may comprise a pair of barcode sequences. In alternative embodiments, the ATAC DNA barcode oligonucleotide and the gene expression barcode oligonucleotide may be the same.

As used herein, the term “GEM well” or “GEM group” refers to a set of partitioned cells (i.e., Gel beads-in-Emulsion or GEMs) from a single 10× Chromium™ Chip channel. One or more sequencing libraries can be derived from a GEM well.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches. In various embodiments within the disclosure, the term adapter can refer to customized strands of nucleic acid base pairs created to bind with specific nucleic acid sequences, e.g., sequences of DNA.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “macromolecule” or “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “sample,” as used herein, generally refers to a “biological sample” of a subject. The sample may be obtained from a tissue of a subject. The sample may be a cell sample. A cell may be a live cell. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears. In some embodiments, the term “sample” can refer to a cell or nuclei suspension extracted from a single biological source (blood, tissue, etc.).

The sample may comprise any number of macromolecules, for example, cellular macromolecules. The sample maybe or may include one or more constituents of a cell, but may not include other constituents of the cell. An example of such cellular constituents is a nucleus or an organelle. The sample may be or may include DNA, RNA, organelles, proteins, or any combination thereof. The sample may be or include a chromosome or other portion of a genome. The sample may be or may include a bead (e.g., a gel bead) comprising a cell or one or more constituents from a cell, such as DNA, RNA, a cell nucleus, organelles, proteins, or any combination thereof, from the cell. The sample may be or may include a matrix (e.g., a gel or polymer matrix) comprising a cell or one or more constituents from a cell, such as DNA, RNA, a cell nucleus, organelles, proteins, or any combination thereof, from the cell.

As used herein, the term “PCR duplicates” refers to duplicates created during PCR amplification. During PCR amplification of the fragments, each unique fragment that is created may result in multiple read-pairs sequenced with near identical barcodes and sequence data. These duplicate reads are identified computationally, and collapsed into a single fragment record for downstream analysis.

### Single Cell Sequencing and Data Analysis Workflows

### Single Cell Sequencing Workflow

In accordance with various embodiments, sequencing data may be obtained by single-cell sequencing methods such as droplet-based single cell sequencing as discussed below, sci-CAR (Single-cell Combinatorial Indexing Chromatin Accessibility and mRNA; Cao J et al., Joint profiling of chromatin accessibility and gene expression in thousands of single cells. Science 361: 1380-1385 (2018), incorporated by reference in their entirety), SNARE-seq (Single-Nucleus Chromatin Accessibility and mRNA Expression sequencing; Chen et al., High-throughput sequencing of the transcriptome and chromatin accessibility in the same cell. Nat Biotechnol 37, 1452-1457 (2019), incorporated by reference in their entirety), or a combination of.

Any known single cell sequencing methods can be used to provide single cell sequencing data for feature linkage methods and systems in various embodiments. In various embodiments, single cells can be separated into partitions such as droplets or wells, wherein each partition comprises a single cell with a known identifier like a barcode. The barcode can be attached to a support, for example, a bead, such as a solid bead or a gel bead.

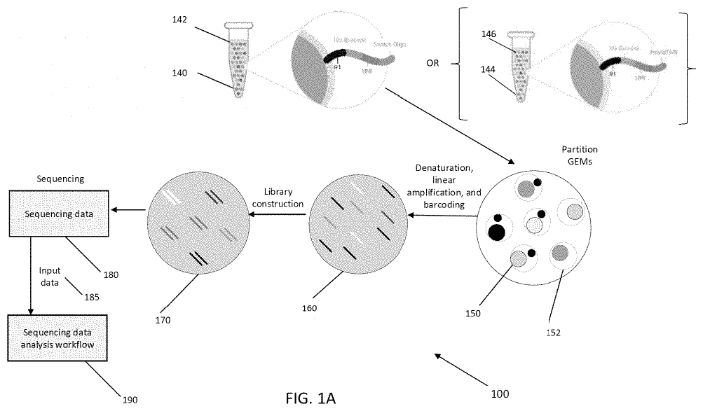

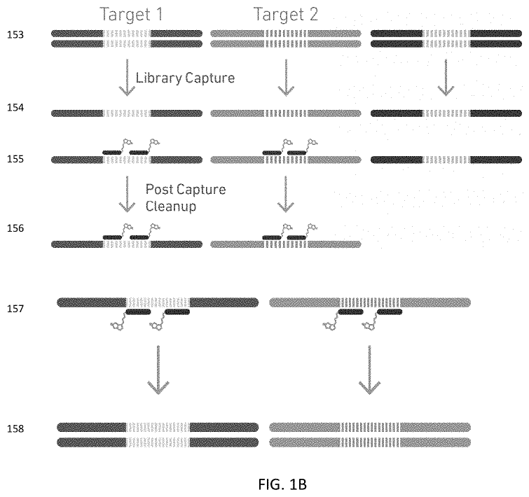

In accordance with various embodiments, a general schematic workflow is provided in FIGS. 1A and 1B to illustrate a non-limiting example process for using single cell sequencing technology to generate single cell sequencing data. Such sequencing data can be used for identifying genome-wide differential accessibility of gene regulatory elements or gene expression analysis in accordance with various embodiments. The workflow can include various combinations of features, whether it be more or less features than that illustrated in FIGS. 1A and 1B. As such, FIGS. 1A and 1B simply illustrate one example of a possible workflow.

Gel Beads-In-EMulsion (GEMs) Generation

The workflow 100 provided in FIG. 1A begins with Gel beads-in-EMulsion (GEMs) generation. The bulk cell suspension containing the cells is mixed with a gel beads solution 140 or 144 containing a plurality of individually barcoded gel beads 142 or 146. In various embodiments, this step results in partitioning the cells into a plurality of individual GEMs 150, each including a single cell, and a barcoded gel bead 142 or 146. This step also results in a plurality of GEMs 152, each containing a barcoded gel bead 142 or 146 but no nuclei. Detail related to GEM generation, in accordance with various embodiments disclosed herein, is provided below. Further details can be found in U.S. Pat. Nos. 10,343,166 and 10,583,440, US Published Application Nos. US20180179590A1, US20190367969A1, US20200002763A1, and US20200002764A1, and Published International PCT Application No. WO 2019/040637, each of which is incorporated herein by reference in its entirety.

In various embodiments, GEMs can be generated by combining barcoded gel beads, individual cells, and other reagents or a combination of biochemical reagents that may be necessary for the GEM generation process. Such reagents may include, but are not limited to, a combination of biochemical reagents (e.g., a master mix) suitable for GEM generation and partitioning oil. The barcoded gel beads 142 or 146 of the various embodiments herein may include a gel bead attached to oligonucleotides containing (i) an Illumina® P5 sequence (adapter sequence), (ii) a 16 nucleotide (nt) 10× Barcode, and (iii) a Read 1 (Read 1N) sequencing primer sequence. It is understood that other adapter, barcode, and sequencing primer sequences can be contemplated within the various embodiments herein.

In various embodiments, GEMS are generated by partitioning the cells using a microfluidic chip. To achieve single cell resolution per GEM, the cells can be delivered at a limiting dilution, such that the majority (e.g., ˜90-99%) of the generated GEMs do not contain any cells, while the remainder of the generated GEMs largely contain a single cell.

Barcoding Nucleotide Fragments

The workflow 100 provided in FIG. 1A further includes lysing the cells and barcoding the RNA molecules or fragments for producing a plurality of uniquely barcoded single-stranded nucleic acid molecules or fragments. Upon generation of the GEMs 150, the gel beads 142 or 146 can be dissolved releasing the various oligonucleotides of the embodiments described above, which are then mixed with the RNA molecules or fragments resulting in a plurality of uniquely barcoded single-stranded nucleic acid molecules or fragments 160 following a nucleic acid extension reaction, e.g., reverse transcription of mRNA to cDNA, within the GEMs 150. Detail related to generation of the plurality of uniquely barcoded single-stranded nucleic acid molecules or fragments 160, in accordance with various embodiments disclosed herein, is provided below.

In various embodiments, upon generation of the GEMs 150, the gel beads 142 or 146 can be dissolved, and oligonucleotides of the various embodiments disclosed herein, containing a capture sequence, e.g., a poly(dT) sequence or a template switch oligonucleotide (TSO) sequence, a unique molecular identifier (UMI), a unique 10× Barcode, and a Read 1 sequencing primer sequence can be released and mixed with the RNA molecules or fragments and other reagents or a combination of biochemical reagents (e.g., a master mix necessary for the nucleic acid extension process). Denaturation and a nucleic acid extension reaction, e.g., reverse transcription, within the GEMs can then be performed to produce a plurality of uniquely barcoded single-stranded nucleic acid molecules or fragments 160. In various embodiments herein, the plurality of uniquely barcoded single-stranded nucleic acid molecules or fragments 160 can be 10× barcoded single-stranded nucleic acid molecules or fragments. In one non-limiting example of the various embodiments herein, a pool of ˜750,000, 10× barcodes are utilized to uniquely index and barcode nucleic acid molecules derived from the RNA molecules or fragments of each individual cell.

Accordingly, the in-GEM barcoded nucleic acid products of the various embodiments herein can include a plurality of 10× barcoded single-stranded nucleic acid molecules or fragments that can be subsequently removed from the GEM environment and amplified for library construction, including the addition of adaptor sequences for downstream sequencing. In one non-limiting example of the various embodiments herein, each such in-GEM 10× barcoded single-stranded nucleic acid molecule or fragment can include a unique molecular identifier (UMI), a unique 10× barcode, a Read 1 sequencing primer sequence, and a fragment or insert derived from an RNA fragment of the cell, e.g., cDNA from an mRNA via reverse transcription. Additional adaptor sequence may be subsequently added to the in-GEM barcoded nucleic acid molecules after the GEMs are broken.

In various embodiments, after the in-GEM barcoding process, the GEMs 150 are broken and pooled barcoded nucleic acid molecules or fragments are recovered. The 10× barcoded nucleic acid molecules or fragments are released from the droplets, i.e., the GEMs 150, and processed in bulk to complete library preparation for sequencing, as described in detail below. In various embodiments, following the amplification process, leftover biochemical reagents can be removed from the post-GEM reaction mixture. In one embodiment of the disclosure, silane magnetic beads can be used to remove leftover biochemical reagents. Additionally, in accordance with embodiments herein, the unused barcodes from the sample can be eliminated, for example, by Solid Phase Reversible Immobilization (SPRI) beads.

Library Construction

The workflow 100 provided in FIG. 1A further includes a library construction step. In the library construction step of workflow 100, a library 170 containing a plurality of double-stranded DNA molecules or fragments are generated. These double-stranded DNA molecules or fragments can be utilized for completing the subsequent sequencing step. Detail related to the library construction, in accordance with various embodiments disclosed herein, is provided below.

In accordance with various embodiments disclosed herein, an Illumina® P7 sequence and P5 sequence (adapter sequences), a Read 2 (Read 2N) sequencing primer sequence, and a sample index (SI) sequence(s) (e.g., i7 and/or i5) can be added during the library construction step via PCR to generate the library 170, which contains a plurality of double stranded DNA fragments. In accordance with various embodiments herein, the sample index sequences can each comprise of one or more oligonucleotides. In one embodiment, the sample index sequences can each comprise of four to eight or more oligonucleotides. In various embodiments, when analyzing the single cell sequencing data for a given sample, the reads associated with all four of the oligonucleotides in the sample index can be combined for identification of a sample. Accordingly, in one non-limiting example, the final single cell gene expression analysis sequencing libraries contain sequencer compatible double-stranded DNA fragments containing the P5 and P7 sequences used in Illumina® bridge amplification, sample index (SI) sequence(s) (e.g., i7 and/or i5), a unique 10× barcode sequence, and Read 1 and Read 2 sequencing primer sequences.

Various embodiments of single cell sequencing technology within the disclosure can at least include platforms such as One Sample, One GEM Well, One Flowcell; One Sample, One GEM well, Multiple Flowcells; One Sample, Multiple GEM Wells, One Flowcell; Multiple Samples, Multiple GEM Wells, One Flowcell; and Multiple Samples, Multiple GEM Wells, Multiple Flowcells platform. Accordingly, various embodiments within the disclosure can include sequence dataset from one or more samples, samples from one or more donors, and multiple libraries from one or more donors.

### Targeted Gene Enrichment by Hybridization Capture

FIG. 1B depicts an example of a workflow for generating a targeted sequencing library using a hybridization capture approach. As shown, step 153 starts with obtaining a library of double stranded barcoded nucleic acid molecules from single cells (e.g., by partitioning single cells into droplets or wells with barcoding reagents including beads having nucleic acid barcode molecules) is denatured to provide single stranded molecules in step 154. To generate a targeted library using the single stranded molecules, a plurality of oligonucleotide probes designed to cover a panel of selected genes is provided. Each gene in the panel is represented by a plurality of labeled (e.g., biotinylated) oligonucleotide probes, which is allowed to hybridize to the single stranded molecules in step 155 to enrich for genes of interest (e.g. Target 1 and Target 2). To allow for capture, step 155 further includes the addition of supports (e.g., beads) that comprise a molecule having affinity for the labels on each labeled oligonucleotide probe. In one embodiment, the oligonucleotide label comprises biotin and the supports comprise streptavidin beads. Following hybridization capture, cleanup steps 156 and 157 are performed (e.g., one or more washing steps to remove unhybridized or off-target library fragments). Captured library fragments are then subjected to nucleic acid extension/amplification to generate a final targeted library for sequencing in step 158. This workflow allows the generation of targeted libraries from gene expression assays. In general, this workflow may be used to enrich any library of fragments having inserts or targets (light gray bar regions) that represent genes, e.g., cDNA transcribed from mRNA of single cells. It should be appreciated, however, that although the description above describes targeted gene enrichment through the use of hybridization capture probes, the methods disclosed herein can also work with other targeted gene enrichment techniques.

The workflow 100 provided in FIG. 1 further includes a sequencing step. In this step, the library 170 can be sequenced to generate a plurality of sequencing data 180. The fully constructed library 170 can be sequenced according to a suitable sequencing technology, such as a next-generation sequencing protocol, to generate the sequencing data 180. In various embodiments, the next-generation sequencing protocol utilizes the Illumina® sequencer for generating the sequencing data. It is understood that other next-generation sequencing protocols, platforms, and sequencers such as, e.g., MiSeq™, NextSeq™ 500/550 (High Output), HiSeq 2500™ (Rapid Run), HiSeq™ 3000/4000, and NovaSeq™, can be also used with various embodiments herein.

Sequencing Data Input and Data Analysis Workflow

The workflow 100 provided in FIG. 1 further includes a sequencing data analysis workflow 190. With the sequencing data 180 in hand, the data can then be output, as desired, and used as an input data 185 for the downstream sequencing data analysis workflow 190 for targeted gene expression analysis, in accordance with various embodiments herein. Sequencing the single cell libraries produces standard output sequences (also referred to as the “sequencing data”, “sequence data”, or the “sequence output data”) that can then be used as the input data 185, in accordance with various embodiments herein. The sequence data contains sequenced fragments (also interchangeably referred to as “fragment sequence reads”, “sequencing reads” or “reads”), which in various embodiments include RNA sequences of the targeted RNA fragments containing the associated 10× barcode sequences, adapter sequences, and primer oligo sequences.

The various embodiments, systems and methods within the disclosure further include processing and inputting the sequence data. A compatible format of the sequencing data of the various embodiments herein can be a FASTQ file. Other file formats for inputting the sequence data is also contemplated within the disclosure herein. Various software tools within the embodiments herein can be employed for processing and inputting the sequencing output data into input files for the downstream data analysis workflow. One example of a software tool that can process and input the sequencing data for downstream data analysis workflow is the cellranger-atac mkfastq tool within the Cell Ranger™ Targeted Gene Expression analysis pipeline (or the scRNA equivalent Cell Ranger™ analysis tool). It is understood that, various systems and methods with the embodiments herein are contemplated that can be employed to independently analyze the inputted single cell targeted gene expression analysis sequencing data for studying cellular gene expression, in accordance with various embodiments.

### Single Cell Sequencing Data Analysis Workflow

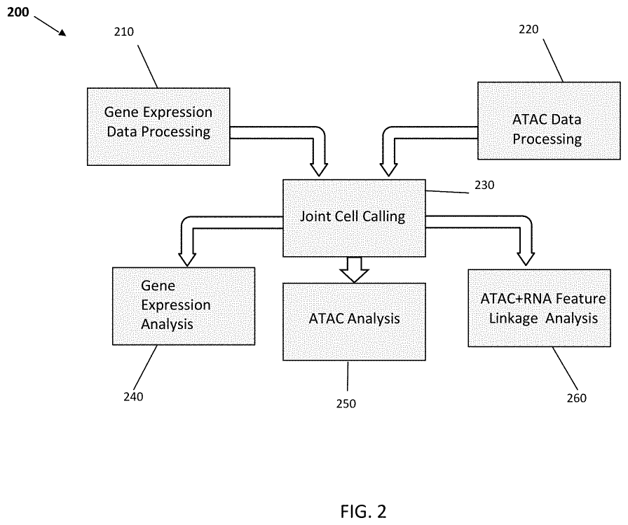

In accordance with various embodiments, a general schematic workflow is provided in FIG. 2 to illustrate a non-limiting example process of a sequencing data analysis workflow for analyzing the single cell sequencing data for gene expression analysis and the single cell ATAC sequencing data for identifying genome-wide differential accessibility of gene regulatory elements. The workflow can include various combinations of features, whether it be more or less features than that illustrated in FIG. 2. As such, FIG. 2 simply illustrates one example of a possible sequencing data analysis workflow.

FIG. 2 provides an example schematic workflow 200, which is an expansion of the sequencing data analysis workflow 190 of FIG. 1, in accordance with various embodiments. It should be appreciated that the methodologies described in the workflow 200 of FIG. 2 and accompanying disclosure can be implemented independently of the methodologies for generating single cell sequencing data described in FIG. 1. Therefore, FIG. 2 can be implemented independently of the sequencing data generating workflow as long as it is capable of sufficiently analyzing single cell sequencing data for gene expression analysis and identifying genome-wide differential accessibility of gene regulatory elements in accordance with various embodiments.

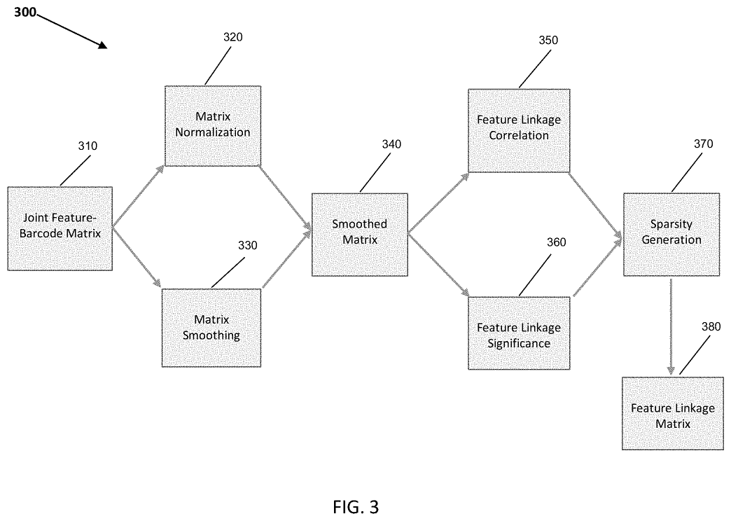

In various embodiments, the example data analysis workflow 200 can include one or more of the following analysis steps, a gene expression data processing step 210, an ATAC data processing step 220, a joint cell calling step 230, a gene expression analysis step 240, an ATAC analysis step 250, and an ATAC and RNA analysis step 260 (which may be described in more detail in FIG. 3)

Not all the steps within the disclosure of FIG. 2 need to be utilized as a group. Therefore, some of the steps within FIG. 2 are capable of independently performing the necessary data analysis as part of the various embodiments disclosed herein. Accordingly, it is understood that, certain steps within the disclosure can be used either independently or in combination with other steps within the disclosure, while certain other steps within the disclosure can only be used in combination with certain other steps within the disclosure. Further, one or more of the steps or filters described below, presumably defaulted to be utilized as part of the computational pipeline for analyzing both the gene expression sequencing data and the single cell ATAC sequencing data, can also not be utilized per user input. It is understood that the reverse is also contemplated. It is further understood that additional steps for analyzing the sequencing data generated by the single cell sequencing workflow are also contemplated as part of the computational pipeline within the disclosure.

Gene Expression Data Processing

The gene expression data processing step 210 can comprise processing the barcodes in the single cell sequencing data set for fixing the occasional sequencing error in the barcodes so that the sequenced fragments can be associated with the original barcodes, thus improving the data quality.

The barcode processing step can include checking each barcode sequence against a “whitelist” of correct barcode sequences. The barcode processing step can further include counting the frequency of each whitelist barcode. The barcode processing step can also include various barcode correction steps as part of the various embodiments disclosed herein. For example, one may attempt to correct the barcodes that are not included on the whitelist by finding all the whitelisted barcodes that are within 2 differences (Hamming distance <=2) of the observed sequence, and then scoring them based on the abundance of that barcode in the read data and quality value of the incorrect bases. As another example, an observed barcode that is not present in the whitelist can be corrected to a whitelist barcode if it has >90% probability of being the real barcode based.

The gene expression data processing 210 can further comprise aligning the read sequences (also referred to as the “reads”) to a reference sequence. In the alignment step of the various embodiments herein, a reference-based analysis is performed by aligning the read sequences (also referred to as the “reads”) to a reference sequence. The reference sequence for the various embodiments herein can include a reference transcriptome sequence (including genes and introns) and its associated genome annotations, which include gene and transcript coordinates. The reference transcriptome sequence and annotations of various embodiments herein can be obtained from reputable, well-established consortia, including but not limited to NCBI, GENCODE, Ensembl, and ENCODE. In various embodiments, the reference sequence can include single species and/or multi-species reference sequences. In various embodiments, systems and methods within the disclosure can also provide pre-built single and multi-species reference sequences. In various embodiments, the pre-built reference sequences can include information and files related to regulatory regions including, but not limited to, annotation of promoter, enhancer, CTCF binding sites, and DNase hypersensitivity sites. In various embodiments, systems and methods within the disclosure can also provide building custom reference sequences that are not pre-built.

Various embodiments herein can be configured to correct for sequencing errors in the UMI sequences, before UMI counting. Reads that were confidently mapped to the transcriptome can be placed into groups that share the same barcode, UMI, and gene annotation. If two groups of reads have the same barcode and gene, but their UMIs differ by a single base (i.e., are Hamming distance 1 apart), then one of the UMIs was likely introduced by a substitution error in sequencing. In this case, the UMI of the less-supported read group is corrected to the UMI with higher support.

After grouping the reads by barcode, UMI (possibly corrected), and gene annotation, if two or more groups of reads have the same barcode and UMI, but different gene annotations, the gene annotation with the most supporting reads is kept for UMI counting, and the other read groups can be discarded. In case of a tie for maximal read support, all read groups can be discarded, as the gene cannot be confidently assigned.

After these two filtering steps, each observed barcode, UMI, gene combination is recorded as a UMI count in an unfiltered feature-barcode matrix, which contains every barcode from fixed list of known-good barcode sequences. This includes background and cell associated barcodes. The number of reads supporting each counted UMI is also recorded in the molecule info file.

The step 210 can further comprise annotating the individual cDNA fragment reads as exonic, intronic, intergenic, and by whether they align to the reference genome with high confidence. In various embodiments, a fragment read is annotated as exonic if at least a portion of the fragment intersects an exon. In various embodiments, a fragment read is annotated as intronic if it is non-exonic and intersects an intron. The annotation process can be determined by the alignment method and its parameters/settings as performed, for example, using the STAR aligner.

The step 210 can further comprise unique molecule processing to better identify certain subpopulations such as for example, low RNA content cells, a unique molecule processing step can be performed prior to cell calling. For low RNA content cells, such a step is important, particularly when low RNA content cells are mixed into a population of high RNA content cells. The unique molecule processing can include a high content (e.g., RNA content) capture step and a low content capture step.

ATAC Data Processing

The ATAC data processing step 220 can comprise processing the barcodes in the single cell ATAC sequencing data for fixing the occasional sequencing error in the barcodes so that the sequenced fragments can be associated with the original barcodes, thus improving the data quality.

The barcode processing step can include checking each barcode sequence against a “whitelist” of correct barcode sequences. The barcode processing step can further include counting the frequency of each whitelist barcode. The barcode processing step can also include various barcode correction steps as part of the various embodiments disclosed herein. For example, one may attempt to correct the barcodes that are not included on the whitelist by finding all the whitelisted barcodes that are within 2 differences (Hamming distance <=2) of the observed sequence, and then scoring them based on the abundance of that barcode in the read data and quality value of the incorrect bases. As another example, an observed barcode that is not present in the whitelist can be corrected to a whitelist barcode if it has >90% probability of being the real barcode based.

The ATAC data processing step 220 can further comprise aligning the read sequences (also referred to as the “reads”) to a reference sequence. One of more sub-steps can be utilized for trimming off adapter sequences, primer oligo sequences, or both in the read sequence before the read sequence is aligned to the reference genome.

The ATAC data processing step 220 can further comprise marking sequencing and PCR duplicates and outputting high quality de-duplicated fragments. One or more sub-steps can be employed for identifying duplicate reads, such as sorting aligned reads by 5′ position to account for transposition event and identifying groups of read-pairs and original read-pair. The process may further include filters that, when activated in various embodiments herein, can determine whether a fragment is mapped with MAPQ>30 on both reads (i.e., includes a barcode overlap for reads with mapping quality below 30), not mitochondrial, and not chimerically mapped.

The ATAC data processing step 220 can comprise a peak calling analysis that includes counting cut sites in a window around each base-pair of the genome and thresholding it to find regions enriched for open chromatin. Peaks are regions in the genome enriched for accessibility to transposase enzymes. Only open chromatin regions that are not bound by nucleosomes and regulatory DNA-binding proteins (e.g., transcription factors) are accessible by transposase enzymes for ATAC sequencing. Therefore, the ends of each sequenced fragment of the various embodiments herein can be considered to be indicative of a region of open chromatin. Accordingly, the combined signal from these fragments can be analyzed in accordance with various embodiments herein to determine regions of the genome enriched for open chromatin, and thereby, to understand the regulatory and functional significance of such regions. Therefore, using the sites as determined by the ends of the fragments in the position-sorted fragment file (e.g., the fragments.tsv.gz file) described above, the number of transposition events at each base-pair along the genome can be counted. In one embodiment within the disclosure, the cut sites in a window around each base-pair of the genome is counted.

Joint Cell Calling Analysis

The joint cell calling analysis step 230 can comprise a cell calling analysis that includes associating a subset of barcodes observed in both the single cell gene expression library and the single cell ATAC library to the cells loaded from the sample. Identification of these cell barcodes can allow one to then analyze the variation and quantification in data at a single cell resolution.

The process may further include correction of gel bead artifacts, such as gel bead multiples (where a cell shares more than one barcoded gel bead) and barcode multiplets (which occurs when a cell associated gel bead has more than one barcode). In some embodiments, the steps associated with cell calling and correction of gel bead artifacts are utilized together for performing the necessary analysis as part of the various embodiments herein.

In accordance with various embodiments, the record of mapped high-quality fragments that passed all the filters of the various embodiments disclosed in the steps above and were indicated as a fragment in the fragment file (e.g., the fragments.tsv file), are recorded. With the peaks determined in the peak calling step disclosed herein, the number of fragments that overlap any peak regions, for each barcode, can be utilized to separate the signal from noise, i.e., to separate barcodes associated with cells from non-cell barcodes. It is to be understood that such method of separation of signal from noise works better in practice as compared to naively using the number of fragments per barcode.

Various methods, in accordance with various embodiments herein, can be employed for joint cell calling. In various embodiments, the joint cell calling can be performed in at least two steps. In the first step of cell calling of the various embodiments herein, the barcodes that have fraction of fragments overlapping called peaks lower than the fraction of genome in peaks are identified. When this first step is employed in the cell calling process of the various embodiments herein, the peaks are padded by 2000 bp on both sides so as to account for the fragment length for this calculation.

Gene Expression Analysis

The gene expression analysis step 240 can comprise generating a feature-barcode matrix that summarizes that gene expression counts per each cell. The feature-barcode matrix can include only detected cellular barcodes. The generation of the feature-barcode matrix can involve compiling the valid non-filtered UMI counts per gene (e.g., output from the ‘Unique Molecule Processing’ step discussed herein) from each cell-associated barcode (e.g., output from the ‘Cell Calling step discussed above) together into the final output count matrix, which can then be used for downstream analysis steps.

The gene expression analysis step 240 can comprise various dimensionality reduction, clustering, t-SNE and UMAP projection tools. Dimensionality reduction tools of the various embodiments herein are utilized to reduce the number of random variables under consideration by obtaining a set of principal variables. In accordance with various embodiments herein, clustering tools can be utilized to assign objects of the various embodiments herein to homogeneous groups (called clusters) while ensuring that objects in different groups are not similar. T-SNE and UMAP projection tools of the various embodiments herein can include an algorithm for visualization of the data of the various embodiments herein. In accordance with various embodiments, systems and methods within the disclosure can further include dimensionality reduction, clustering and t-SNE and UMAP projection tools. In some embodiments, the analysis associated with dimensionality reduction, clustering, and t-SNE and UMAP projection for visualization are utilized together for performing the necessary analysis as part of the various embodiments herein. Various analysis tools for dimensionality reduction include Principal Component Analysis (PCA), Latent Semantic Analysis (LSA), and Probabilistic Latent Semantic Analysis (PLSA), clustering, and t-SNE and UMAP projection for visualization that allow one to group and compare a population of cells with another.

In some embodiments, the systems and methods within the disclosure are directed to identifying differential gene expression. As the data is sparse at single cell resolution, dimensionality reduction in accordance with various embodiments herein can be performed to cast the data into a lower dimensional space.

In accordance with various embodiments, the gene expression analysis step 240 can comprise a differential expression analysis that performs differential analysis to identify genes whose expression is specific to each cluster, Cell Ranger tests, for each gene and each cluster, whether the in-cluster mean differs from the out-of-cluster mean.

ATAC Analysis

The ATAC analysis step 250 can comprise determining the peak-barcode matrix. In accordance with various embodiments, at step 250, a raw peak-barcode matrix can be generated first, which is a count matrix consisting of the counts of fragment ends (or cut sites) within each peak region for each barcode. This raw peak-barcode matrix captures the enrichment of open chromatin per barcode. The raw matrix can then be filtered to consist only of cell barcodes by filtering out the non-cell barcodes from the raw peak-barcode matrix, which can then be used in the various dimensionality reduction, clustering and visualization steps of the various embodiments herein.

The ATAC analysis step 250 can comprise various dimensionality reduction, clustering and t-SNE projection tools, similar to as described above in step 240.

The ATAC analysis step 250 can comprise annotating the peaks by performing gene annotations and discovering transcription factor-motif matches on each peak. It is contemplated that peak annotation can be employed with subsequent differential analysis steps within various embodiments of the disclosure. Various peak annotation procedures and parameters are contemplated and are discussed in detail below.

Peaks are regions enriched for open chromatin, and thus have potential for regulatory function. It is therefore understood that observing the location of peaks with respect to genes can be insightful. Various embodiments herein, e.g., bedtools closest −D=b, can be used to associate each peak with genes based on closest transcription start sites (TSS) that are packaged within the reference. In accordance with some embodiments within the disclosure, a peak is associated with a gene if the peak is within 600 bases upstream or 100 bases downstream of the TSS. Additionally, in accordance with some embodiments within the disclosure, genes can be associated to putative distal peaks that are much further from the TSS and are less than 100 kb upstream or downstream from the ends of the transcript. This association can be adopted by companion visualization software of the various embodiments herein, e.g., Loupe Cell Browser. In another embodiment, this association can be used to construct and visualize derived features such as promoter-sums that can pool together counts from peaks associated with a gene.

The ATAC analysis step 250 can further comprise a transcription factor (TF) motif enrichment analysis. TF motif enrichment analysis includes generating a TF-barcode matrix consisting of the peak-barcode matrix (i.e., pooled cut-site counts for peaks) having a TF motif match, for each motif and each barcode. It is contemplated that the TF motif enrichment can then be utilized for subsequent analysis steps, such as differential accessibility analysis, within various embodiments of the disclosure. Detail related to TF motif enrichment analysis is provided below.

The ATAC analysis step 250 can further comprise a differential accessibility analysis that performs differential analysis of TF binding motifs and peaks for identifying differential gene expression between different cells or groups of cells. Various algorithms and statistical models within the disclosure, such as a Negative Binomial (NB2) generalized linear model (GLM), can be employed for the differential accessibility analysis.

ATAC and RNA Feature Linkage Analysis

The ATAC and RNA analysis step 260 can comprise a feature linkage analysis for detecting correlations between pairs of genomic features detected in each of a plurality of cells, for example, between open chromatin regions and genes from single cell datasets. Such correlations can be denoted as feature linkages or linkage correlations and can be used for inferring enhancer-gene targeting relationships and constructing transcriptional networks. More details for feature linkage analysis will provided in FIG. 3 below.

In various embodiments, joint data from the joint cell calling step 230 can be further processed by the ATAC and RNA analysis step 260 to identify correlations and a significance of the correlations between the single cell gene expression library and the single cell ATAC library.

The features with strong linkage correlations are considered to be “co-expressed” and enrich for a shared regulatory mechanism. For example, the accessibility of an enhancer and the expression of its target gene can display a very synchronized differential pattern across a heterogeneous population of cells. A highly accessible enhancer leads to an elevated level of transcription factor (TF) binding, which in turn leads to elevated (or repressed) gene expression. On the other hand, when the enhancer is inaccessible, no TF can bind to the enhancer, and thus transcription activation is at minimum, which leads to reduced target gene expression.

### Feature Linkage Analysis Workflow

In accordance with various embodiments, a general schematic workflow 300 is provided in FIG. 3 to illustrate a non-limiting example process of a feature linkage analysis workflow for feature linkage analysis. The workflow 300 can include various combinations of features, whether it be more or less features than that illustrated in FIG. 3. As such, FIG. 3 simply illustrates one example of a possible workflow for conducting feature linkage analysis.

FIG. 3 provides a schematic workflow 300 for conducting feature linkage analysis. It should be appreciated that the methodologies described in the workflow 300 of FIG. 3 and accompanying descriptions can be implemented independently of the methodologies for generating single cell gene expression sequencing data or single ATAC sequencing data described in general. Therefore, FIG. 3 can be implemented independently of a sequencing data generating workflow as long as it is capable of sufficiently analyzing single cell sequencing data sets for feature linkage analysis.

Moreover, the data analysis workflow can include one or more of the analysis steps illustrated in FIG. 3. Not all the steps within the disclosure of FIG. 3 need to be utilized as a group. Therefore, some of the steps within FIG. 3 are capable of independently performing the necessary data analysis as part of the various embodiments disclosed herein. Accordingly, it is understood that, certain steps within the disclosure can be used either independently or in combination with other steps within the disclosure, while certain other steps within the disclosure can only be used in combination with certain other steps within the disclosure. Further, one or more of the steps described below, presumably defaulted to be utilized as part of the computational pipeline, can also not be utilized per user input. It is understood that the reverse is also contemplated. It is further understood that additional steps for analyzing the generated sequencing data are also contemplated as part of the computational pipeline within the disclosure.

Joint Feature-Barcode Matrix

In step 310, joint feature-barcode matrix may be generated and received. The joint feature-barcode matrix can be generated by gene expression data processing step 210 and ATAC data processing steps 220. For example, the joint cell barcode matrix may comprise the counts of fragment ends (cut sites) within each peak region for each barcode and the counts of UMIs for each barcode.

Matrix Normalization

In step 320, the joint feature-barcode matrix may be normalized to generate a normalized matrix. The normalization may reduce the bias introduced by the variance of totals signals per single cell. The total signals per cell, alternatively referred as depth, can be the sum of unique molecular identifiers (UMIs) for gene expression or the sum of total cut sites in ATAC.

Previous methods for normalization will create a strong artifact for feature linkage analysis, so a depth-adaptive negative binomial distribution model may be used to overcome this defect. Normalization may comprise selecting genomic features detected in each of a plurality of cells within a pre-set size of genomic window, for example, 100 kb, 200 kb, 300 kb, 400 kb, 500 kb, 600 kb, 700 kb, 800 kb, 900 kb, 1 Mb, 1.5 Mb, 2 Mb, or any intermediate ranges or values therefrom.

Normalization may further comprise using a depth adaptive negative binomial distribution model to model molecular counts of the joint feature-barcode matrix, in which the mean of the distribution for every genomic feature is assumed to vary linearly with the library size for each cell. The negative binomial distribution is a probability distribution that is used with discrete random variables. This type of distribution concerns the number of trials that must occur in order to have a predetermined number of successes. In various embodiments, the depth adaptive negative binomial distribution model may be applied to at least two data types, including, but not limited to, both gene expression data and ATAC data. For example, normalized matrix count \hat{x}ᵢⱼ is a standardized value of raw count xᵢⱼ based on a non-limiting exemplary formula as shown below:

$${l}_{j} = \frac{{x}_{ij}}{{\sum }_{i} {\sum }_{j} {x}_{ij}}$$

$${\hat{μ}}_{ij} = \frac{{x}_{ij}}{{l}_{j}}$$

$${\hat{r}}_{i} = MLE ( {x}_{i} . )$$

$$E [ {x}_{ij} ] = {\hat{μ}}_{ij}$$

$$Var [ {x}_{ij} ] =$$

$$= \frac{{x}_{ij} - E [ {x}_{ij} ]}{\sqrt{Var [ {x}_{ij} ]}}$$

Where xᵢⱼ is entry of the feature-barcode matrix for feature i and cell j and \hat{x}ᵢⱼ is the normalized value for feature i and cell j. “μ hat” and “r hat” represent the negative binomial mean and dispersion.

Matrix Smoothing

In step 330, the joint feature-barcode matrix may be smoothed by K-nearest neighbors (KNN) distance and Gaussian kernel to generate a cell-cell similarity matrix.

Due to the sparsity of single-cell data, especially the cut sites count in peaks, it is most likely that one will not detect signals of a peak and a gene at the same time in one cell when both are expected to have high expression levels. As a result, direct computation of correlation or other measures of dependence on the raw counts between two genomic features detected in each of a plurality of cells may fail to generate any meaningful value that distinguishes highly co-expressed features from the rest.

To overcome this barrier, smoothing may be performed so the value of a feature in a given cell is enhanced by “borrowing” values of the same feature from “neighboring” cells. Here, neighboring cells describe a population of cells whose gene expression profile or ATAC profile share a high similarity, i.e., a low distance. For example, the distance is an Euclidean distance. The Euclidean distance or Euclidean metric is the “ordinary” straight-line distance between two points in Euclidean space.

The high similarity may be determined by applying a K-nearest neighbor algorithm called “Ball-Tree” on the principal component analysis (PCA)-reduced dimension. For example, the ball tree nearest-neighbor algorithm examines nodes in depth-first order, starting at the root. During the search, the algorithm maintains a max-first priority queue (often implemented with a heap), denoted Q here, of the K nearest points encountered so far. Principal component analysis (PCA) refers to a main linear technique for dimensionality reduction and performs a linear mapping of the data to a lower-dimensional space in such a way that the variance of the data in the low-dimensional representation is maximized.

Smoothing comprises “borrowing” information from neighboring cells. In various embodiments, the information “borrowing” can be achieved by a weighted summation of signals of all a predetermined number of neighboring cells using K-nearest neighbors distance (for example, K=30). K may be selected to be 10, 20, 30, 40, 50, 60, 70, 80, 90, 100 or any intermediate ranges or values, depending on how many cells are in a given dataset. For example, if more than 10,000 cells are available, a larger K value (K=50) might be selected.

In various embodiments, the cell-to-cell similarity matrix may determine the smoothing weights. The smoothing weights may be determined as the Euclidean distance based on the gene expression principal components, such that weight Wij is only positive if cells i and j are neighbors and there are no self-edges.

Additionally and alternatively, to avoid over-smoothing, raw distances can be normalized using a Gaussian kernel:

$$K ( x , {x}^{′} ) = exp ( - \frac{{‖ x - {x}^{′} ‖}^{2}}{2 {σ}^{2}} )$$

In certain embodiments, based on the use of a Gaussian kernel, smoothing weights are high only when two cells have a highly similar gene expression profile and quickly decays to zero when the similarity between cells decreases. The ‘kernel’ for smoothing, defines the shape of the function that is used to take the average of the neighboring points. A Gaussian kernel is a kernel with the shape of a Gaussian (normal distribution) curve.

After smoothing, putative co-expressed features showed a much strong correlation pattern compared to randomly selected pairs of features.

Smoothed Matrix

In step 340, a smoothed matrix may be generated by the normalized matrix from step 320 and the cell-cell similarity matrix from step 330. For example, the smoothed matrix may be generated by multiplying the normalized matrix with the cell-cell similarity matrix.

Feature Linkage Correlations

In step 350, feature linkage correlations may be generated. Linkage correlation is the direct measure of the strength of the linkage, with the value bound by [−1, 1]. The sign of the correlation indicates a positive or negative association. It provides a very interpretable measure of the linkage strength.

For example, feature linkage correlations may be generated by computing a Pearson correlation coefficient between two genomic features detected in each of a plurality of cells as the linkage correlation after smoothing.

The Pearson's correlation coefficient r, for vector X and Y of the same length, referred as Pearson correlation, may be computed as:

$${r}_{x y} = \frac{n \sum {x}_{i} {y}_{i} - \sum {x}_{i} \sum {y}_{i}}{\sqrt{n \sum {x}_{i}^{2} - {( \sum {x}_{i} )}^{2}} \sqrt{n \sum {y}_{i}^{2} - {( \sum {y}_{i} )}^{2}}} .$$

Where {(x1, y1), (x2, y2), . . . , (xn, yn)} are the paired data of X and Y, i is a cell number (1, 2, 3, . . . , N), and n is the sample size.

The workflow 300 can comprise, at step 370, generating feature linkage significances. In various embodiments, feature linkage significances may be generated as a probability score.

Significance of feature linkage provides measures of statistical uncertainty for feature linkage inference and offers more contrast of strong linkages relative to weak linkages. Significance may be generated by determining a local correlation value for a linkage between at least two genomic features detected in each of a plurality of cells and transform the value to a Gaussian random variable. This method allows for hypothesis testing.

For example, linkage significance is computed using a modified algorithm based on improvements and extensions of local correlation from Hotspot (DeTomaso et al., DeTomaso, D., & Yosef, N. (2020). Identifying Informative Gene Modules Across Modalities of Single Cell Genomics. BioRxiv, 2020.02.06.937805).

$${H}_{xy} = {w}_{ij} ( {x}_{i} {y}_{j} + {y}_{i} {x}_{j} )$$

$$E ( {H}_{xy} ) = 0$$

$$E ( {H}_{xy}^{2} ) = {( {\sum }_{i}^{N} {\sum }_{j ∈ N ( i )} {w}_{ij} {x}_{j} )}^{2}$$

$$= \frac{{H}_{ij} - E ( H )}{\sqrt{E ( {H}^{2} ) - {( E ( H ) )}^{2}}} = \frac{{w}_{ij} ( {x}_{i} {y}_{j} + {y}_{i} {x}_{j} )}{{( {\sum }_{i}^{N} {\sum }_{j ∈ N ( i )} {w}_{ij} {x}_{j} )}^{2}}$$

Specifically, computation of Hxy and E(Hxy2) was significantly accelerated by converting a loop-based procedure in DeTomaso et al. into a matrix multiplication-based procedure. In this matrix multiplication, local correlation of a N pairs of features (for example, 10,000 pairs of features), denoted as the Z score “Zxy hat”, can be generated in one step of operation instead of a loop of N operations (for example, 10,000 operations).

Additionally and alternatively, the local correlation Z score may be extended to a hypothesis-testing framework to generate a probability score. Because the Z score follows a Gaussian distribution of mean 0 and variance 1 based on the normalization step as described above, it can be converted to a probability score and subject to multiple testing correction.

The resulting value is a false discovery rate for whether a given pair of features x and y are significantly correlated.

Sparsity Generation

The workflow 300 can comprise, at step 370, sparsity generation. A sparse statistical model is one in which only a relatively small number of parameters (or predictors) play an important role. Because the number of computable linkages is quadratic of the number of features, and it is expected that majority of computable linkages are not biologically significant, it is natural to expect sparsity in the inference of feature linkage.

Since most feature linkages are not significant, a subset of linkages that have a significance lower than a pre-set threshold may be filtered out and the sparsified linkage matrix may be used for better interpretation. Thresholding may be selected based on feature significance. For a particular example, a thresholding method may be used, in which linkages with significance <5 are removed from the linkage matrix. The threshold can be determined based on an analysis of continuously down-sampling reads and comparing of the decay of linkage significance and correlation. For example, significance=5 may have the best balance of linkage strength and stability against down-sampling. In various embodiments, thresholding may use a feature significance threshold, such as significance more than or equal to 4, 4.5, 5, 5.5, 6 or any intermediate ranges or values derived therefrom for selecting for feature linkages. In additional and alternative embodiments, thresholding may be set using a value of correlation, for example, feature linkages with a correlation value more than 0.2, 0.25, 0.3, 0.35, 0.4, 0.45, 0.5 or any intermediate values or ranges may be selected and set as a threshold for selecting for feature linkages.

Several sparsity-generating strategies may be used. For example, the sparsity-generation may use thresholding, which is to exclude linkages with a pre-set threshold on correlation or significance. Thresholding may be a particular example of sparsity-generating strategy based on its simplicity, interpretability, and good consistency against differential expression.

In additional and alternative embodiments, the sparsity-generation may use Gaussian graphical models (GGM). A GGM is an undirected graph in which each edge represents the pairwise correlation between two variables conditioned against the correlations with all other variables (also denoted as partial correlation coefficients). GGMs have a simple interpretation in terms of linear regression techniques. When regressing two random variables X and Y on the remaining variables in the data set, the partial correlation coefficient between X and Y can be determined by the Pearson correlation of the residuals from both regressions. Intuitively speaking, we remove the (linear) effects of all other variables on X and Y and compare the remaining signals. If the variables are still correlated, the correlation is directly determined by the association of X and Y and not mediated by the other variables.

Several flavors of GGM-based methods have been tested and can be used, including, but not being limited to, graphical lasso, relaxed graphical lasso, sparse estimation of covariance, and sparse Steinian covariance estimation. The benefit of GGM is that it has a strong statistics framework and allows linkage-specific regularization. However, GGM based on optimizing the precision matrix creates false negative, in which strong linkages can be erroneously determined to be zero. GGM that optimizing the covariance matrix may need to be used to improve GGM-based sparsity generation.

Feature Linkage Matrix

The workflow 300 can comprise, at step 380, generating a feature linkage matrix after sparsity generation for downstream analysis.

### Feature Linkage Analysis Methods

In various embodiments, methods are provided for feature linkage analysis. The methods can be implemented via computer software or hardware. The methods can also be implemented on a computing device/system that can include a combination of engines for feature linkage analysis. In various embodiments, the computing device/system can be communicatively connected to one or more of a data source, sample analyzer (e.g., a genomic sequence analyzer), and display device via a direct connection or through an internet connection.

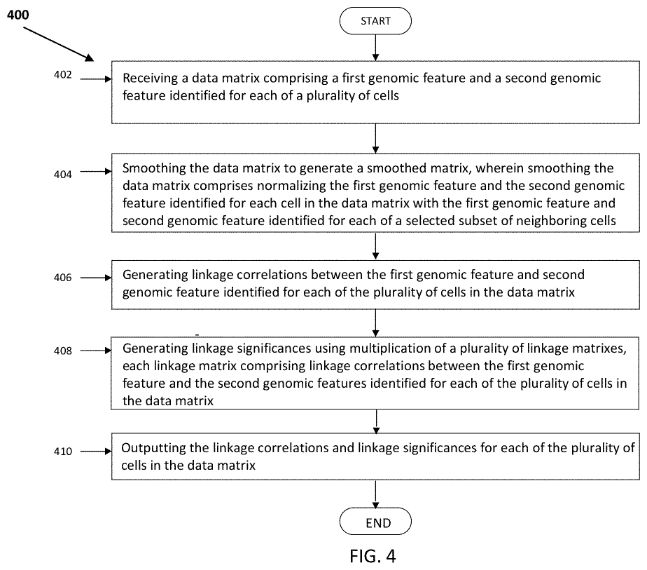

Referring now to FIG. 4, a flowchart illustrating a non-limiting example method 400 for feature linkage analysis is disclosed, in accordance with various embodiments. The method can comprise, at step 402, receiving a data matrix comprising at least two genomic features detected in each of a plurality of cells. For example, at least two genomic features can be gene expression features (such as genes and mRNA) and assay for transposase-accessible chromatin (ATAC) features (such as open chromatin regions or accessible chromatin regions). For example, the data matrix may be a joint feature-barcode matrix that comprises data of both cut sites and UMIs for each barcode. In additional and alternative embodiments, the data matrix may be generated from single-cell sequencing as discussed above, sci-CAR or SNARE-seq, or a combination thereof.

The method can comprise, at step 404, smoothing the data matrix to generate a smoothed matrix, wherein smoothing the data matrix comprises normalizing the first genomic feature and the second genomic feature identified for each cell in the data matrix with the first and second genomic features from a subset of neighboring cells. Normalizing the data matrix may comprise using a depth-adaptive negative binomial distribution model to model molecular counts of the data matrix, such as joint feature-barcode matrix.

The method can comprise, at step 406, generating linkage correlations between the first genomic feature and second genomic feature identified for each of the plurality of cells in the data matrix. For example, feature linkage correlations may be generated by computing a Pearson correlation coefficient between two genomic features as the linkage correlation after smoothing.

The method can comprise, at step 408, generating linkage significances of the linkage correlations of pairs of the first and second genomic features identified for each of the plurality of cells in the data matrix. In various embodiments, feature linkage significances may be generated as a probability score. For example, the feature linkage significances can be generated by using multiplication of a plurality of linkage matrixes. Each linkage matrix can comprise linkage correlations of pairs of the first and second genomic features identified for each of the plurality of cells in the data matrix.

In additional and alternative embodiments, the feature linkage significances may be generated using a matrix multiplication. In this matrix multiplication, local correlation of a N pairs of features (for example, 10,000 pairs of features), denoted as the Z score “Zxy hat”, can be generated in one step of operation instead of a loop of N operations (for example, 10,000 operations).

The method can comprise, at step 410, outputting the linkage correlations and linkage significances.

### Feature Linkage Analysis Systems

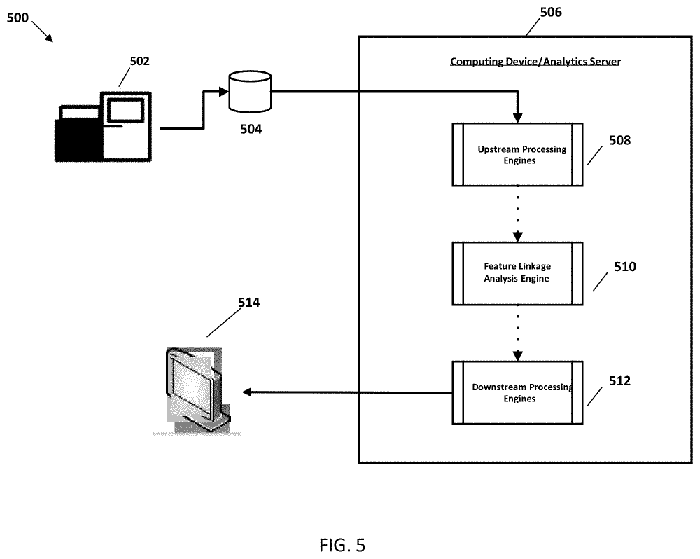

In accordance with various embodiments, FIG. 5 illustrates a non-limiting example system for feature linkage analysis, in accordance with various embodiments. The system 500 includes a genomic sequence analyzer 502, a data storage unit 504, a computing device/analytics server 506, and a display 514.

The genomic sequence analyzer 502 can be communicatively connected to the data storage unit 504 by way of a serial bus (if both form an integrated instrument platform) or by way of a network connection (if both are distributed/separate devices). The genomic sequence analyzer 502 can be configured to process, analyze and generate two or more genomic sequence datasets from a sample, such as the single cell gene expression libraries and the single cell ATAC libraries of the various embodiments herein. Each fragment in the single cell gene expression libraries includes an associated barcode and unique molecular identifier sequence (i.e., UMI). In various embodiments, the genomic sequence analyzer 502 can be a next-generation sequencing platform or sequencer such as the Illumina® sequencer, MiSeq™, NextSeq™ 500/550 (High Output), HiSeq 2500™ (Rapid Run), HiSeq™ 3000/4000, and NovaSeq.

In various embodiments, the generated genomic sequence datasets can then be stored in the data storage unit 504 for subsequent processing. In various embodiments, one or more raw genomic sequence datasets can also be stored in the data storage unit 504 prior to processing and analyzing. Accordingly, in various embodiments, the data storage unit 504 can be configured to store one or more genomic sequence datasets, e.g., the genomic sequence datasets of the various embodiments herein that includes a plurality of fragment sequence reads with their associated barcodes and unique identifier sequences from the single cell gene expression libraries and the single cell ATAC libraries. In various embodiments, the processed and analyzed genomic sequence datasets can be fed to the computing device/analytics server 506 in real-time for further downstream analysis.

In various embodiments, the data storage unit 504 is communicatively connected to the computing device/analytics server 506. In various embodiments, the data storage unit 504 and the computing device/analytics server 506 can be part of an integrated apparatus. In various embodiments, the data storage unit 504 can be hosted by a different device than the computing device/analytics server 506. In various embodiments, the data storage unit 504 and the computing device/analytics server 506 can be part of a distributed network system. In various embodiments, the computing device/analytics server 506 can be communicatively connected to the data storage unit 504 via a network connection that can be either a “hardwired” physical network connection (e.g., Internet, LAN, WAN, VPN, etc.) or a wireless network connection (e.g., Wi-Fi, WLAN, etc.). In various embodiments, the computing device/analytics server 506 can be a workstation, mainframe computer, distributed computing node (part of a “cloud computing” or distributed networking system), personal computer, mobile device, etc.

In various embodiments, the computing device/analytics sever 506 is configured to host one or more upstream data processing engines 508, a feature linkage analysis engine 510, and one or more downstream data processing engines 512.

Examples of upstream data processing engines 508 can include, but are not limited to: alignment engine, cell barcode processing engine (for correcting sequencing barcode sequencing errors), alignment engine (for aligning the fragment sequence reads to a reference genome), duplicate marking engine (identifying duplicate reads by sorting reads), peak calling engine (for counting cut sites in a window around each base-pair of the genome and thresholding it to find regions enriched for open chromatin), annotation engine (for annotating each of the aligned fragment sequence reds with relevant information), a joint cell calling engine (for grouping fragment sequence reads and gene expression sequence reads as being from a unique cell), feature barcode matrix engine (for creating a feature barcode matrix), peak barcode matrix engine (for creating a peak barcode matrix), joint feature barcode matrix engine (for creating a joint feature barcode matrix), etc.

The feature linkage analysis engine 510 can be configured to receive genomic sequence datasets such as a data matrix comprising at least two genomic features identified for each of a plurality of cells stored in the data storage unit 504. For example, at least two genomic features can be gene expression features (such as genes and mRNA) and assay for transposase-accessible chromatin (ATAC) features (such as accessible chromatin regions or open chromatin regions, e.g., enhancers or promoters). For example, the data matrix may be a joint feature-barcode matrix that comprises data of both cut sites and UMIs for each barcode. In additional and alternative embodiments, the data matrix may be generated from single-cell sequencing as discussed above, sci-CAR or SNARE-seq, or a combination thereof. In various embodiments, the feature linkage analysis engine 510 can be configured to receive processed and analyze genomic sequence datasets from the genomic sequence analyzer 502 in real-time.

In various embodiments, the feature linkage analysis engine 510 can be configured to smooth the data matrix to generate a smoothed matrix. In various embodiments, smoothing the data matrix comprises normalizing the first genomic feature and the second genomic feature identified for each cell in the data matrix with the first and second genomic features identified for each of a selected subset of neighboring cells. Normalizing the data matrix may comprise using a depth adaptive negative binomial distribution model to model molecular counts of the data matrix, such as joint feature-barcode matrix.

In various embodiments, the feature linkage analysis engine 510 can be configured to generate linkage correlations between the first genomic feature and second genomic feature identified for each of the plurality of cells in the data matrix. For example, feature linkage correlations may be generated by computing a Pearson correlation coefficient between two genomic features identified for each of the plurality of cells in the data matrix as the linkage correlation after smoothing.

In various embodiments, the feature linkage analysis engine 510 can be configured to generate linkage significances of the linkage correlations of pairs of the first and second genomic features identified for each of the plurality of cells in the data matrix. In various embodiments, feature linkage significances may be generated as a probability score. For example, the feature linkage significances can be generated by using multiplication of a plurality of linkage matrixes. Each linkage matrix can comprise linkage correlations of pairs of the first and second genomic features identified for each of the plurality of cells in the data matrix.

The identified subset of cell then be further processed by one or more downstream data processing engines 512. Examples of downstream data processing engines 512 can include, but are not limited to: secondary analysis engine (including dimensionality reduction, clustering, t-SNE projection), enhancer discovery engine, transcription factor engine (for mapping transcription factors to peaks), topological domain engine, etc.

In various embodiments, the secondary analysis engine can use feature linkages as new genomic features for each single cell. For example, if a cell has signal for both features of a given feature linkage, it can be assigned as 1, otherwise 0. The new binary features can be used for dimensionality reduction and clustering of cells.

In various embodiments, the enhancer discovery engine can intersect and compare feature linkages to bulk Chromatin Immunoprecipitation Sequencing (ChIP-Seq) (histone modification marks, CCCTC-binding factor (CTCF), etc) of Hi-C data. Strong linkages with overlap to these epigenetic features (for example, H3K27Ac) identified in Chip-Seq can be predicted as enhancers.

In various embodiments, the transcription factor engine can match transcription factor motifs in peaks involved in a peak-gene feature linkage. Matched transcription factor motifs can be further filtered by whether that transcription factor gene is expressed based on the gene expression data. After matching, the transcription factor engine can connect the transcription factor and the gene involved in the feature linkage. The transcription factor engine can construct a transcription factor network using these connections.

In various embodiments, the topological domain engine can group linked features based on locality into a super group. The topological domain engine can use the super group to compare with topological domains inferred from chromatin conformation capture assays, such as Hi-C, and to construct genomic interaction topological domains.

After the downstream processing has been performed, an output of the results can be displayed as a result or summary on a display or client terminal 514 that is communicatively connected to the computing device/analytics server 506. In various embodiments, the display or client terminal 514 can be a client computing device. In various embodiments, the display or client terminal 514 can be a personal computing device having a web browser (e.g., INTERNET EXPLORER™, FIREFOX™, SAFARI™, etc.) that can be used to control the operation of the genomic sequence analyzer 502, data storage unit 504, upstream data processing engines 508, feature linkage analysis engine 510, and the downstream data processing engines 512.

It should be appreciated that the various engines can be combined or collapsed into a single engine, component or module, depending on the requirements of the particular application or system architecture. In various embodiments engines 508/510/512 can comprise additional engines or components as needed by the particular application or system architecture.

### Examples

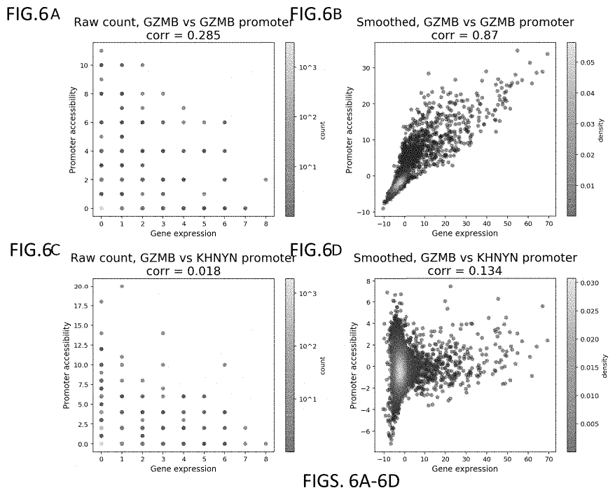

FIGS. 6A-6D are graphs depicting an effect in which matrix smoothing improves interpretability of the linkage correlation. Examples of strong feature linkage (FIG. 6A, FIG. 6B) and weak feature linkage (FIG. 6C, FIG. 6D) from a PBMC dataset with 3,799 cells are shown. Raw counts are represented on a gray scale based on the number of barcodes that share the same raw counts (FIG. 6A, FIG. 6C). Note that more than 3,000 cells have raw counts of (0, 0). Smoothed values are represented on a gray scale as the density in the 2-D scatter plot (FIG. 6C, FIG. 6D).

GZMB (Granzyme B) is a known natural killer (NK) and CD8 T cell marker in PBMC, and its expression is highly enriched in NK and CD8 cytotoxic T cells. As the GZMB promoter displays significant NK and CD8 T cell-specific accessibility, the GZMB promoter and the gene were determined to have strong feature linkage (FIGS. 6A-6B). However, because of the sparsity in the count data, more than 80% of the cells (3094 out of 3799) had zero count in both GZMB gene expression and GZMB promoter. Moreover, more than 40% (355 out of 877) of annotated CD8/NK cells had zeros in both features (FIG. 6A). The correlation of the raw counts between GZMB gene expression and GZMB promoter accessibility was 0.285 and was visually difficult to interpret as one of the strongest feature linkages in PBMCs (FIG. 6A).

After smoothing, the correlation of GZMB gene expression and GZMB promoter accessibility was 0.87, with a clear visual pattern of correlation (FIG. 6B). The promoter of KHNYN, a nearby gene, does not have NK or CD8 T cell-specific differential accessibility. Instead, it has comparable accessibility in all major PBMC cell types. Hence, the linkage between KGNYN promoter and GZMB gene was determined to be weak, as evident from the low correlation value (FIGS. 6C-6D).

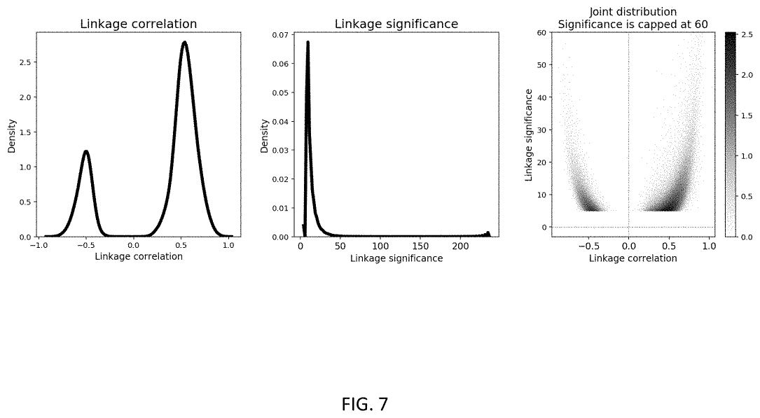

FIG. 7 are plots depicting distributions for linkage correlation and significance for a 5 k peripheral blood mononuclear cell (PBMC) dataset, in accordance with various embodiments. The left plot shows linkage correlation with density plotted on the y-axis and linkage correlation plotted on the x-axis. The middle plot shows linkage significance with density plotted on the y-axis and linkage significance plotted on the x-axis. The right plot shows a joint distribution of linkage correlation and linkage significance with linkage significance plotted on the y-axis and linkage correlation plotted on the x-axis. The joint distribution shows thresholding significance automatically enriched strong correlations with almost no exceptions.

### Computer-Implemented Systems

In various embodiments, the methods comprise receiving a multi-genomic feature sequence dataset for feature linkage analysis and can be implemented via computer software or hardware. That is, as depicted in FIG. 5, the methods disclosed herein can be implemented on a computing device 506 that includes upstream data processing engines 508, a feature linkage analysis engine 510 and downstream data processing engines 512. In various embodiments, the computing device 506 can be communicatively connected to a data storage unit 504 and a display device 514 via a direct connection or through an internet connection.

It should be appreciated that the various engines depicted in FIG. 5 can be combined or collapsed into a single engine, component or module, depending on the requirements of the particular application or system architecture. Moreover, in various embodiments, the upstream data processing engines 508, feature linkage analysis engine 510 and downstream data processing engines 512 can comprise additional engines or components as needed by the particular application or system architecture.

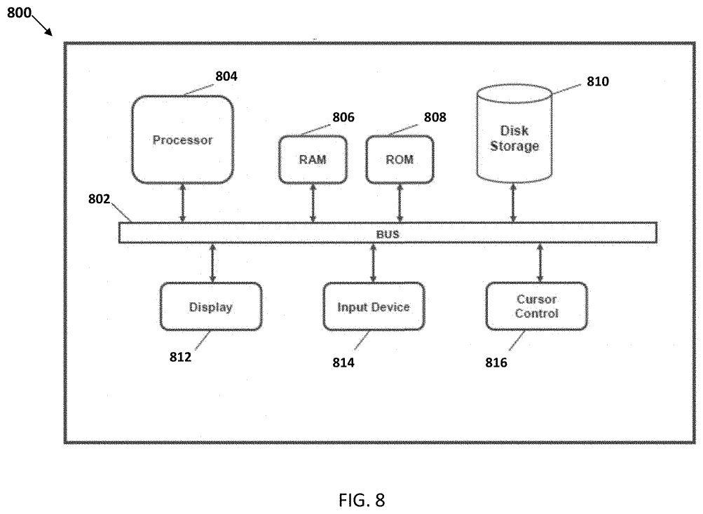

FIG. 8 is a block diagram illustrating a computer system 800 upon which embodiments of the present teachings may be implemented. In various embodiments of the present teachings, computer system 800 can include a bus 802 or other communication mechanism for communicating information and a processor 804 coupled with bus 802 for processing information. In various embodiments, computer system 800 can also include a memory, which can be a random-access memory (RAM) 806 or other dynamic storage device, coupled to bus 802 for determining instructions to be executed by processor 804. Memory can also be used for storing temporary variables or other intermediate information during execution of instructions to be executed by processor 804. In various embodiments, computer system 800 can further include a read only memory (ROM) 808 or other static storage device coupled to bus 802 for storing static information and instructions for processor 804. A storage device 810, such as a magnetic disk or optical disk, can be provided and coupled to bus 802 for storing information and instructions.

In various embodiments, computer system 800 can be coupled via bus 802 to a display 812, such as a cathode ray tube (CRT) or liquid crystal display (LCD), for displaying information to a computer user. An input device 814, including alphanumeric and other keys, can be coupled to bus 802 for communication of information and command selections to processor 804. Another type of user input device is a cursor control 816, such as a mouse, a trackball or cursor direction keys for communicating direction information and command selections to processor 804 and for controlling cursor movement on display 812. This input device 814 typically has two degrees of freedom in two axes, a first axis (i.e., x) and a second axis (i.e., y), that allows the device to specify positions in a plane. However, it should be understood that input devices 814 allowing for 3-dimensional (x, y and z) cursor movement are also contemplated herein.

Consistent with certain implementations of the present teachings, results can be provided by computer system 800 in response to processor 804 executing one or more sequences of one or more instructions contained in memory 806. Such instructions can be read into memory 806 from another computer-readable medium or computer-readable storage medium, such as storage device 810. Execution of the sequences of instructions contained in memory 806 can cause processor 804 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 804 for execution. Such a medium can take many forms, including but not limited to, non-volatile media, volatile media, and transmission media. Examples of non-volatile media can include, but are not limited to, dynamic memory, such as memory 806. Examples of transmission media can include, but are not limited to, coaxial cables, copper wire, and fiber optics, including the wires that comprise bus 802.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 804 of computer system 800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 800 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 800, whereby processor 804 would execute the analyses and determinations provided by these engines, subject to instructions provided by any one of, or a combination of, memory components 806/808/810 and user input provided via input device 814.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### Recitation of Embodiments

Embodiment 1: A method for generating linkage correlations and linkage significances between a first genomic feature and a second genomic feature identified for each of a plurality of cells, the method comprising receiving a data matrix comprising a first genomic feature and a second genomic feature identified for each of a plurality of cells; smoothing the data matrix to generate a smoothed matrix, wherein smoothing the data matrix comprises normalizing the first genomic feature and the second genomic feature identified for each cell in the data matrix with the first genomic feature and second genomic feature identified for each of a selected subset of neighboring cells; generating linkage correlations between the first genomic feature and second genomic feature identified for each of the plurality of cells in the data matrix; generating linkage significances using multiplication of a plurality of linkage matrixes, each linkage matrix comprising linkage correlations between the first genomic feature and the second genomic features identified for each of the plurality of cells in the data matrix; and outputting the linkage correlations and linkage significances for each of the plurality of cells in the data matrix.

Embodiment 2: The method of Embodiment 1, wherein the first genomic feature comprises genes.

Embodiment 3: The method of Embodiment 2, wherein the second genomic feature comprises open chromatin regions.

Embodiment 4: The method of Embodiment 3, wherein the open chromatic regions comprise regulatory elements that affects expression of genes.

Embodiment 5: The method of any one of Embodiments 1 to 4, wherein smoothing the data matrix further comprises selecting the first and second genomic features identified for each of the plurality of cells in the data matrix with a pre-set genomic window.

Embodiment 6: The method of any one of Embodiments 1 to 5, wherein smoothing the data matrix further comprises generating a normalized matrix using depth-adaptive negative binomial normalization for the first genomic feature and second genomic feature identified for each of the plurality of cells in the data matrix.

Embodiment 7: The method of Embodiment 6, wherein smoothing the data matrix further comprises generating a cell-cell similarity matrix by a weighted summation of the first genomic feature and second genomic feature identified for each of the selected subset of neighboring cells of the data matrix, wherein weights are determined using a Gaussian kernel.

Embodiment 8: The method of Embodiment 7, wherein smoothing the data matrix comprises multiplying the cell-cell similarity matrix with the normalized matrix to generate the smoothed matrix.

Embodiment 9: The method of any one of Embodiments 1 to 8, wherein generating linkage correlations comprises obtaining a Pearson correlation between the first and second genomic features identified for each of the plurality of cells in the data matrix.

Embodiment 10: The method of any one of Embodiments 1 to 9, wherein generating linkage significances comprises obtaining a probability score of the linkage correlations.

Embodiment 11: The method of any one of Embodiments 1 to 10, further comprising validating the linkage correlations.

Embodiment 12: The method of any one of Embodiments 1 to 11, further comprising filtering out a subset of linkage correlations lower than a pre-set threshold to output remaining linkage correlations.

Embodiment 13: A non-transitory computer-readable medium storing computer instructions that, when executed by a computer, cause the computer to perform a method for generating linkage correlations and linkage significances between a first genomic feature and a second genomic feature identified for each of a plurality of cells, the method comprising receiving a data matrix comprising the first genomic feature and the second genomic feature identified for each of a plurality of cells; smoothing the data matrix to generate a smoothed matrix, wherein smoothing the data matrix comprises normalizing the first genomic feature and the second genomic feature identified for each cell in the data matrix with the first genomic feature and second genomic feature identified for each of a selected subset of neighboring cells; generating linkage correlations between the first genomic feature and second genomic feature identified for each of the plurality of cells in the data matrix; generating linkage significances using multiplication of a plurality of linkage matrixes, each linkage matrix comprising linkage correlations between the first genomic feature and the second genomic features identified for each of the plurality of cells in the data matrix; and outputting the linkage correlations and linkage significances for each of the plurality of cells in the data matrix.

Embodiment 14: The non-transitory computer-readable medium of Embodiment 13, wherein smoothing the data matrix further comprises selecting the first and second genomic features identified for each of the plurality of cells in the data matrix with a pre-set genomic window.

Embodiment 15: The non-transitory computer-readable medium of any one of Embodiments 13 to 14, wherein smoothing the data matrix further comprises generating a normalized matrix using depth-adaptive negative binomial normalization for the first genomic feature and second genomic feature identified for each of the plurality of cells in the data matrix.

Embodiment 16: The non-transitory computer-readable medium of Embodiment 15, wherein smoothing the data matrix further comprises generating a cell-cell similarity matrix by a weighted summation of the first genomic feature and second genomic feature identified for each of the selected subset of neighboring cells of the data matrix, wherein weights are determined using a Gaussian kernel.

Embodiment 17: The non-transitory computer-readable medium of Embodiment 16, wherein smoothing the data matrix comprises multiplying the cell-cell similarity matrix with the normalized matrix to generate the smoothed matrix.

Embodiment 18: The non-transitory computer-readable medium of any one of Embodiments 13 to 17, wherein generating linkage correlations comprises obtaining a Pearson correlation between the first and second genomic features identified for each of the plurality of cells in the data matrix.

Embodiment 19: The non-transitory computer-readable medium of any one of Embodiments 13 to 18, wherein generating linkage significances comprises obtaining a probability score of the linkage correlations.

Embodiment 20: The non-transitory computer-readable medium of any one of Embodiments 13 to 19, wherein the method further comprises validating the linkage correlations.

Embodiment 21: The non-transitory computer-readable medium of claim 13, wherein the method further comprises filtering out a subset of linkage correlations lower than a pre-set threshold to output remaining linkage correlations.

Embodiment 22: A system for generating linkage correlations and linkage significances between a first genomic feature and a second genomic feature identified for each of a plurality of cells, comprising: a data store configured to store a data set at least associated with a plurality of cells, wherein the data set comprises molecule counts of at least two genomic features for each cell of a plurality of cells; and a computing device communicatively connected to the data store and configured to receive the data set, the computing device comprising a feature linkage analysis engine configured to receive a data matrix comprising the first genomic feature and the second genomic feature identified for each of a plurality of cells, smooth the data matrix to generate a smoothed matrix, wherein smoothing the data matrix comprises normalizing the first genomic feature and the second genomic feature identified for each cell in the data matrix with the first genomic feature and second genomic feature identified for each of a selected subset of neighboring cells, generate linkage correlations between the first genomic feature and second genomic feature identified for each of the plurality of cells in the data matrix, and generate linkage significances using multiplication of a plurality of linkage matrixes, each linkage matrix comprising linkage correlations between the first genomic feature and the second genomic features identified for each of the plurality of cells in the data matrix; and a display communicatively connected to the computing device and configured to display a report comprising the linkage correlations and linkage significances.

Embodiment 23: The system of Embodiment 22, wherein the first genomic feature comprises genes.

Embodiment 24: The system of any one of Embodiments 22 or 23, wherein the second genomic feature comprises open chromatin regions.

Embodiment 25: The system of any one of Embodiments 22 to 24, wherein smoothing the data matrix further comprises selecting the first and second genomic features identified for each of the plurality of cells in the data matrix with a pre-set genomic window.

Embodiment 26: The system of any one of Embodiments 22 to 25, wherein smoothing the data matrix further comprises generating a normalized matrix using depth-adaptive negative binomial normalization for the first genomic feature and second genomic feature identified for each of the plurality of cells in the data matrix.

Embodiment 27: The system of Embodiment 26, wherein smoothing the data matrix further comprises generating a cell-cell similarity matrix by a weighted summation of the first genomic feature and second genomic feature identified for each of the selected subset of neighboring cells of the data matrix, wherein weights are determined using a Gaussian kernel.

Embodiment 28: The system of Embodiment 27, wherein smoothing the data matrix comprises multiplying the cell-cell similarity matrix with the normalized matrix to generate the smoothed matrix.

Embodiment 29: The system of any one of Embodiments 22 to 28, wherein generating linkage correlations comprises obtaining a Pearson correlation between the first and second genomic features identified for each of the plurality of cells in the data matrix.

Embodiment 30: The system of any one of Embodiments 22 to 29, wherein generating linkage significances comprises obtaining a probability score of the linkage correlations.

Embodiment 31: The system of any one of Embodiments 22 to 30, wherein the feature linkage analysis engine is further configured to validate the linkage correlations.

Embodiment 32: The system of any one of Embodiments 22 to 31, wherein the feature linkage analysis engine is further configured to filter out a subset of linkage correlations lower than a pre-set threshold and to output remaining linkage correlations.

What is claimed:

1. A method for generating linkage correlations and linkage significances between a first genomic feature and a second genomic feature identified for each of a plurality of cells, the method comprising:
receiving a data matrix comprising a first genomic feature and a second genomic feature identified for each of a plurality of cells;
smoothing the data matrix to generate a smoothed matrix, wherein smoothing the data matrix comprises normalizing the first genomic feature and the second genomic feature identified for each cell in the data matrix with the first genomic feature and second genomic feature identified for each of a selected subset of neighboring cells;
generating linkage correlations between the first genomic feature and second genomic feature identified for each of the plurality of cells in the data matrix;
generating linkage significances using multiplication of a plurality of linkage matrixes, each linkage matrix comprising linkage correlations between the first genomic feature and the second genomic features identified for each of the plurality of cells in the data matrix; and
outputting the linkage correlations and linkage significances for each of the plurality of cells in the data matrix.

2. The method of claim 1, wherein the first genomic feature comprises genes.

3. The method of claim 2, wherein the second genomic feature comprises open chromatin regions.

4. The method of claim 3, wherein the open chromatic regions comprise regulatory elements that affect expression of genes.

5. The method of claim 1, wherein smoothing the data matrix further comprises selecting the first and second genomic features identified for each of the plurality of cells in the data matrix with a pre-set genomic window.

6. The method of claim 1, wherein smoothing the data matrix further comprises generating a normalized matrix using depth-adaptive negative binomial normalization for the first genomic feature and second genomic feature identified for each of the plurality of cells in the data matrix.

7. The method of claim 6, wherein smoothing the data matrix further comprises generating a cell-cell similarity matrix by a weighted summation of the first genomic feature and second genomic feature identified for each of the selected subset of neighboring cells of the data matrix, wherein weights are determined using a Gaussian kernel.

8. The method of claim 7, wherein smoothing the data matrix comprises multiplying the cell-cell similarity matrix with the normalized matrix to generate the smoothed matrix.

9. The method of claim 1, wherein generating linkage correlations comprises obtaining a Pearson correlation between the first and second genomic features identified for each of the plurality of cells in the data matrix.

10. The method of claim 1, wherein generating linkage significances comprises obtaining a probability score of the linkage correlations.

11. The method of claim 1, further comprising validating the linkage correlations.

12. The method of claim 1, further comprising filtering out a subset of linkage correlations lower than a pre-set threshold to output remaining linkage correlations.

13. A non-transitory computer-readable medium storing computer instructions that, when executed by a computer, cause the computer to perform a method for generating linkage correlations and linkage significances between a first genomic feature and a second genomic feature identified for each of a plurality of cells, the method comprising:
receiving a data matrix comprising the first genomic feature and the second genomic feature identified for each of a plurality of cells;
smoothing the data matrix to generate a smoothed matrix, wherein smoothing the data matrix comprises normalizing the first genomic feature and the second genomic feature identified for each cell in the data matrix with the first genomic feature and second genomic feature identified for each of a selected subset of neighboring cells;
generating linkage correlations between the first genomic feature and second genomic feature identified for each of the plurality of cells in the data matrix;
generating linkage significances using multiplication of a plurality of linkage matrixes, each linkage matrix comprising linkage correlations between the first genomic feature and the second genomic features identified for each of the plurality of cells in the data matrix; and
outputting the linkage correlations and linkage significances for each of the plurality of cells in the data matrix.

14. The non-transitory computer-readable medium of claim 13, wherein smoothing the data matrix further comprises selecting the first and second genomic features identified for each of the plurality of cells in the data matrix with a pre-set genomic window.

15. The non-transitory computer-readable medium of claim 13, wherein smoothing the data matrix further comprises generating a normalized matrix using depth-adaptive negative binomial normalization for the first genomic feature and second genomic feature identified for each of the plurality of cells in the data matrix.

16. The non-transitory computer-readable medium of claim 15, wherein smoothing the data matrix further comprises generating a cell-cell similarity matrix by a weighted summation of the first genomic feature and second genomic feature identified for each of the selected subset of neighboring cells of the data matrix, wherein weights are determined using a Gaussian kernel.

17. The non-transitory computer-readable medium of claim 16, wherein smoothing the data matrix comprises multiplying the cell-cell similarity matrix with the normalized matrix to generate the smoothed matrix.

18. The non-transitory computer-readable medium of claim 13, wherein generating linkage correlations comprises obtaining a Pearson correlation between the first and second genomic features identified for each of the plurality of cells in the data matrix.

19. The non-transitory computer-readable medium of claim 13, wherein generating linkage significances comprises obtaining a probability score of the linkage correlations.

20. The non-transitory computer-readable medium of claim 13, wherein the method further comprises validating the linkage correlations.

21. The non-transitory computer-readable medium of claim 13, wherein the method further comprises filtering out a subset of linkage correlations lower than a pre-set threshold to output remaining linkage correlations.

22. A system for generating linkage correlations and linkage significances between a first genomic feature and a second genomic feature identified for each of a plurality of cells, comprising:
a data store configured to store a data set at least associated with a plurality of cells, wherein the data set comprises molecule counts of at least two genomic features for each cell of a plurality of cells; and
a computing device communicatively connected to the data store and configured to receive the data set, the computing device comprising a feature linkage analysis engine configured to
receive a data matrix comprising the first genomic feature and the second genomic feature identified for each of a plurality of cells,
smooth the data matrix to generate a smoothed matrix, wherein smoothing the data matrix comprises normalizing the first genomic feature and the second genomic feature identified for each cell in the data matrix with the first genomic feature and second genomic feature identified for each of a selected subset of neighboring cells,
generate linkage correlations between the first genomic feature and second genomic feature identified for each of the plurality of cells in the data matrix, and
generate linkage significances using multiplication of a plurality of linkage matrixes, each linkage matrix comprising linkage correlations between the first genomic feature and the second genomic features identified for each of the plurality of cells in the data matrix; and
a display communicatively connected to the computing device and configured to display a report comprising the linkage correlations and linkage significances.

23. The system of claim 22, wherein the first genomic feature comprises genes.

24. The system of claim 23, wherein the second genomic feature comprises open chromatin regions.

25. The system of claim 22, wherein smoothing the data matrix further comprises selecting the first and second genomic features identified for each of the plurality of cells in the data matrix with a pre-set genomic window.

26. The system of claim 22, wherein smoothing the data matrix further comprises generating a normalized matrix using depth-adaptive negative binomial normalization for the first genomic feature and second genomic feature identified for each of the plurality of cells in the data matrix.

27. The system of claim 26, wherein smoothing the data matrix further comprises generating a cell-cell similarity matrix by a weighted summation of the first genomic feature and second genomic feature identified for each of the selected subset of neighboring cells of the data matrix, wherein weights are determined using a Gaussian kernel.

28. The system of claim 27, wherein smoothing the data matrix comprises multiplying the cell-cell similarity matrix with the normalized matrix to generate the smoothed matrix.

29. The system of claim 22, wherein generating linkage correlations comprises obtaining a Pearson correlation between the first and second genomic features identified for each of the plurality of cells in the data matrix.

30. The system of claim 22, wherein generating linkage significances comprises obtaining a probability score of the linkage correlations.

31. The system of claim 22, wherein the feature linkage analysis engine is further configured to validate the linkage correlations.

32. The system of claim 22, wherein the feature linkage analysis engine is further configured to filter out a subset of linkage correlations lower than a pre-set threshold and to output remaining linkage correlations.
